(19) United States
(12) Patent Application Publication
(10) Pub. No.: US 2026/0253679 A1
(43) Pub. Date: Aug. 27, 2026

(54) RECORDING MEDIUM, INFORMATION PROCESSING METHOD, AND INFORMATION PROCESSING DEVICE

(71) Applicant: Fujitsu Limited, Kawasaki-shi (JP)

(72) Inventor: Sho TAKEMORI, Yokohama (JP)

(73) Assignee: Fujitsu Limited, Kawasaki-shi (JP)

(21) Appl. No.: 19/542,799

(22) Filed: Feb. 18, 2026

(30) Foreign Application Priority Data
Feb. 27, 2025 (JP) 2025-030451

(51) Int. Cl. G16C 20/20; G06F 17/11; G06F 17/17

(52) U.S. Cl. CPC G16C 20/20; G06F 17/11; G06F 17/17

(57) ABSTRACT

A computer-readable recording medium stores therein a program for causing a computer to execute a process, the process including: calculating a plurality of density matrices respectively corresponding to a plurality of fragments obtained by dividing a structure of a molecule, based on a predetermined parameter by using a first optimization calculation method; and updating the predetermined parameter according to a Newton-Krylov method so as to maintain consistency among the plurality of fragments, when a predetermined exit condition is not satisfied, the predetermined parameter being updated based on the calculated plurality of density matrices and by referring to a Jacobian approximation formula and using a second optimization calculation method having a smaller calculation amount than the first optimization calculation method. The calculating and updating are iteratively executed until the predetermined exit condition is satisfied.

### CROSS REFERENCE TO RELATED APPLICATIONS

This application is based upon and claims the benefit of priority of the prior Japanese Patent Application No. 2025-030451, filed on Feb. 27, 2025, the entire contents of which are incorporated herein by reference.

### FIELD

The embodiments discussed herein are related to a recording medium, an information processing method, and an information processing device.

### BACKGROUND

Conventionally, in the field of drug discovery, material development, or the like, there is a quantum chemical calculation technique for analyzing the structure or properties of a molecule that is a candidate for a drug or a material. In the quantum chemical calculation, for example, the energy of a molecule is calculated. The energy is the ground or excitation energy. Here, in order to reduce the processing amount of the quantum chemical calculation, there is a molecule dividing method of splitting the structure of a molecule into multiple fragments and then calculating energy.

As the related art, for example, there is a molecule dividing method called Bootstrap Embedding (BE) in which a series of processes of calculating a density matrix corresponding to fragments and performing optimization calculation so as to maintain consistency between the fragments is iteratively performed. Further, for example, there is a molecule dividing method called Density Matrix Embedding Theory (DMET) in which an interaction between fragments is treated as a bath orbital. For example, refer to Hong-Zhou Ye, Henry K. Tran, and Troy Van Voorhis, “Bootstrap embedding for large molecular systems.” Journal of Chemical Theory and Computation 16.8 (2020): 5035-5046; and Gerald Knizia and Garnet Kin-Lic Chan, “Density matrix embedding: A simple alternative to dynamical mean-field theory.” Physical review letters 109.18 (2012): 186404.

### SUMMARY

According to an aspect of an embodiment, a computer-readable recording medium stores therein a program for causing a computer to execute a process, the process including: calculating a plurality of density matrices respectively corresponding to a plurality of fragments obtained by dividing a structure of a molecule, based on a predetermined parameter by using a first optimization calculation method; and updating the predetermined parameter according to a Newton-Krylov method so as to maintain consistency among the plurality of fragments, when a predetermined exit condition is not satisfied, the predetermined parameter being updated based on the calculated plurality of density matrices and by referring to a Jacobian approximation formula and using a second optimization calculation method having a smaller calculation amount than the first optimization calculation method. The calculating and updating are iteratively executed until the predetermined exit condition is satisfied.

The object and advantages of the disclosure will be realized and attained by means of the elements and combinations particularly pointed out in the claims.

It is to be understood that both the foregoing general description and the following detailed description are exemplary and explanatory and are not restrictive of the disclosure.

### BRIEF DESCRIPTION OF DRAWINGS

FIG. 1 is an explanatory diagram depicting an example of an information processing method according to an embodiment.

FIG. 2 is an explanatory diagram depicting an example of an information processing system 200.

FIG. 3 is a block diagram depicting an example of a hardware configuration of an information processing device 100.

FIG. 4 is a block diagram depicting an example of a functional configuration of the information processing device 100.

FIG. 5 is an explanatory diagram depicting an example of operation of the information processing device 100.

FIG. 6 is a flowchart depicting an example of a procedure of an overall process.

### DESCRIPTION OF EMBODIMENTS

First problems associated with the conventional techniques are discussed. In the related arts, even when the molecule dividing method is used, it may be difficult to reduce the processing amount of the quantum chemical calculation. For example, there is a problem in that the amount of processing necessary to perform optimization calculation for maintaining consistency between fragments tends to increase.

Embodiments of an information processing device 100, an information processing method, and an recording medium according to the present disclosure will be explained below in detail with reference to the accompanying drawings.

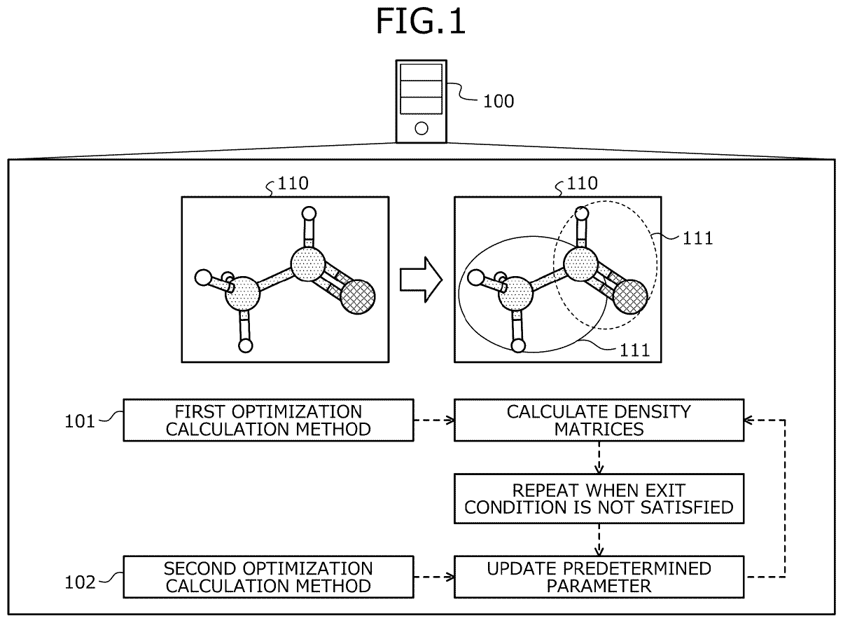

FIG. 1 is an explanatory diagram depicting an example of an information processing method according to an embodiment. The information processing device 100 is a computer for reducing the amount of processing necessary when performing optimization calculation in a process of performing quantum chemical calculation using a molecule dividing method. The information processing device 100 is, for example, a server or a personal computer (PC).

Here, the quantum chemical calculation, for example, uses a specific optimization calculation method to calculate the energy of a molecule. The energy is the ground or excitation energy. The optimization calculation method is, for example, Coupled Cluster (CC), Full Configuration Interaction (FCI), Variational Quantum Eigen-solver (VQE), or the like. CC is, for example, a coupled-cluster calculation with single and double excitations (CCSD).

Here, as the scale of the molecule increases, the throughput of the quantum chemical calculation tends to increase. The scale is the number of atoms forming the molecule, etc. Thus, in order to reduce the processing amount of the quantum chemical calculation, there is a molecule dividing method of dividing the structure of a molecule into multiple fragments and then calculating the energy. Examples of the molecule dividing method include BE and DMET.

BE specifically divides the structure of a molecule into multiple fragments. Specifically, the BE iterates a series of processes of calculating a density matrix corresponding to each fragment and performing optimization calculation to maintain consistency between fragments, based on the density matrix when a predetermined exit condition is not satisfied until the predetermined exit condition is satisfied. The optimization calculation is performed by, for example, Newton's method. Regarding BE, for example, Hong-Zhou Ye, et al, “Bootstrap embedding for large molecular systems” may be referred to.

However, in the related art, even when the molecule dividing method is used, it may be difficult to reduce the processing amount of the quantum chemical calculation. For example, in the case of using BE, there is a problem in that the processing amount tends to increase when optimization calculation for maintaining consistency between fragments is performed, and it is difficult to reduce the processing amount of quantum chemical calculation. Specifically, when the optimization calculation is performed by the Newton method, the processing amount necessary to calculate the Jacobian of an objective function tends to increase.

On the other hand, a first method of calculating the Jacobian of the objective function by the finite difference method using a relatively high-precision optimization method such as CC, FCI, or VQE is conceivable. In the first method, since the optimization method with relatively high accuracy is iteratively performed, the processing amount necessary to calculate the Jacobian of the objective function tends to increase, and the processing amount necessary to perform the optimization calculation tends to increase. For the first method, for example, Hong-Zhou Ye, et al, “Bootstrap embedding for large molecular systems” may be referred to.

In addition, a second method is considered in which the initial value of the Jacobian is obtained by the Hartree-Fock method, and then the optimization calculation is performed by a quasi-Newton method. In the second method, when the Jacobian is updated, a relatively high-precision optimization method is iteratively performed and thus, the amount of processing necessary to perform optimization calculation tends to increase. For the second method, for example, Ye, Hong-Zhou, et al, “Bootstrap embedding for molecules.” Journal of chemical theory and computation 15.8 (2019): 4497-4506 may be referred to.

Further, a third method of approximating the Jacobian by combining the finite difference method and the Krylov method is conceivable. In the third method, since the optimization method with relatively high accuracy is iteratively performed, the processing amount necessary for performing the optimization calculation tends to increase. For the third technique, for example, Knoll, Dana A., and David E. Keyes, “Jacobian-free Newton-Krylov methods: a survey of approaches and applications.” Journal of Computational Physics 193.2 (2004): 357-397 may be referred to.

Therefore, in the present embodiment, an information processing method capable of easily reducing the processing amount necessary for performing optimization calculation in a process of performing quantum chemical calculation using a molecule dividing method will be described.

In FIG. 1, the information processing device 100 may use a first optimization calculation method 101. For example, the information processing device 100 may perform the first optimization calculation method 101 by itself. The first optimization calculation method 101 is, for example, CCSD, FCI, VQE, or the like. The information processing device 100 may use a second optimization calculation method 102 having a smaller calculation amount than the first optimization calculation method 101. For example, the information processing device 100 may perform the second optimization calculation method 102 by itself. The second optimization calculation method 102 is, for example, a second-order Moller-Plesset method.

The information processing device 100 obtains structural information indicating a molecular structure 110. The structural information represents, for example, the position and type of each of one or more atoms forming the molecule. The structural information represents, for example, a connection relationship between atoms. The information processing device 100 obtains the structure information by receiving an input of the structure information based on the operation input of the user.

The information processing device 100 obtains fragment information indicating each fragment 111 among multiple fragments 111 obtained by dividing the molecular structure 110. The fragment information represents, for example, the position and type of each of one or more atoms belonging to the fragment 111. The fragments 111 share, for example, any atom. The information processing device 100 obtains the fragment information by receiving an input of the fragment information based on an operation input of the user, for example.

The information processing device 100 stores a predetermined exit condition. The predetermined exit condition is set in advance by the user, for example. The predetermined exit condition is, for example, that the density matrices respectively corresponding to the fragments 111 match or are similar. The information processing device 100 iteratively executes a series of processes described in (1-1) and (1-2) below until a predetermined exit condition is satisfied.
(1-1) The information processing device 100 calculates a density matrix corresponding to each fragment 111 based on a predetermined parameter using the first optimization calculation method 101. The predetermined parameter includes, for example, a Lagrange multiplier. The predetermined parameter may include, for example, a chemical potential u.
(1-2) When the predetermined exit condition is not satisfied, the information processing device 100 updates the predetermined parameter using the second optimization calculation method 102 so as to maintain the consistency between the fragments 111 with respect to the multiple fragments 111. For example, using the second optimization calculation method 102, the information processing device 100 refers to the Jacobian approximation formula and based on the calculated density matrices, updates the predetermined parameter according to the Newton-Krylov method so as to maintain the consistency between the fragments 111.

Accordingly, the information processing device 100 may easily reduce the processing amount necessary when performing the optimization calculation for updating the predetermined parameter in the process of performing the quantum chemical calculation using the molecule dividing method. Thus, the information processing device 100 may easily reduce the processing amount when performing the quantum chemical calculation.
(1-3) When a predetermined exit condition is satisfied, the information processing device 100 may calculate the energy of the molecule, based on the predetermined parameter last updated and the density matrix last calculated. Accordingly, the information processing device 100 may appropriately calculate the energy of the molecule and complete the quantum chemical calculation. The information processing device 100 may reduce the amount of processing necessary to perform quantum chemical calculation.

Here, while a case in which the information processing device 100 receives input of the structure information based on the operation input of the user has been described, the present disclosure is not limited hereto. For example, the information processing device 100 may obtain the structure information by receiving the structure information from another computer.

Here, while a case in which the information processing device 100 receives input of fragment information has been described, the present disclosure is not limited hereto. For example, the information processing device 100 may obtain fragment information by receiving the fragment information from another computer. For example, the information processing device 100 may obtain fragment information by dividing the molecular structure 110 into the multiple fragments 111 based on structure information and generating the fragment information.

Here, while a case in which functions as the information processing device 100 are realized by a single computer has been described, the present disclosure is not limited hereto. For example, the functions of the information processing device 100 may be realized by cooperation of multiple computers. For example, the functions of the information processing device 100 may be implemented on a cloud.

In the following description, for the sake of simplicity, while the Lagrange multiplier among the predetermined parameters will be focused on, the present disclosure is not limited hereto. For example, the information processing device 100 may perform optimization calculation for updating the chemical potential u among predetermined parameters. Here, the information processing device 100 maintains the consistency between the fragments 111 by updating the chemical potential u so that the total number of electrons of each fragment 111 matches the number of electrons of the molecule.

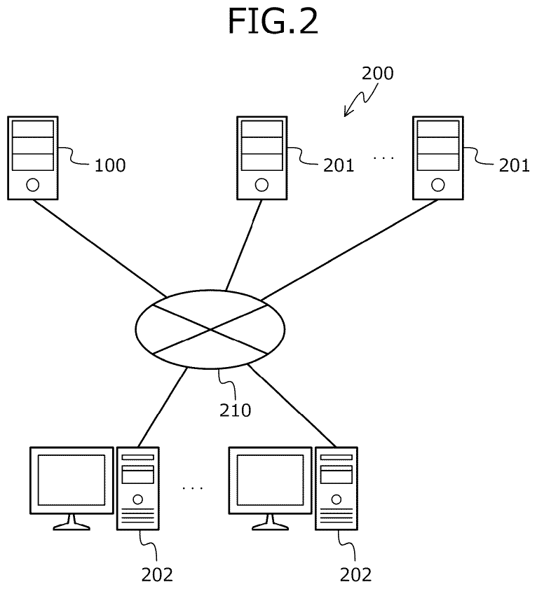

Next, an example of an information processing system 200 to which the information processing device 100 depicted in FIG. 1 is applied will be described with reference to FIG. 2.

FIG. 2 is an explanatory diagram depicting an example of the information processing system 200. In FIG. 2, the information processing system 200 includes the information processing device 100, one or more chemical calculating devices 201, and one or more client devices 202.

In the information processing system 200, the information processing device 100 and each chemical calculating device 201 are connected via a wired or wireless network 210. The network 210 is, for example, a local area network (LAN), a wide area network (WAN), the Internet, or the like. In the information processing system 200, the information processing device 100 and each client device 202 are connected via a wired or wireless network 210.

The information processing device 100 is a computer for dividing the structure of a molecule into multiple fragments. The information processing device 100 obtains a processing request requesting execution of quantum chemical calculation for a molecule of interest by using the molecule dividing method. Quantum chemical calculations include, for example, calculating the energy of the molecule of interest.

The processing request includes, for example, structural information indicating the structure of the molecule of interest. The structural information includes, for example, coordinates of each atom of one or more atoms forming the molecule of interest. The structural information includes, for example, the type of each of one or more atoms forming the molecule of interest. The processing request may include, for example, a division number indicating how many fragments the structure of the molecule of interest is to be divided into. The number of divisions may be set in advance by the user, for example.

The information processing device 100 identifies the structure of the molecule of interest based on the structure information included in the processing request. The information processing device 100 generates fragment information indicating each fragment by dividing the structure of the identified molecule of interest into multiple fragments corresponding to the number of divisions. The information processing device 100 sets an initial value of the Lagrange multiplier. The information processing device 100 extends BE based on the fragment information and performs a quantum chemical calculation for calculating the energy of the molecule of interest.

For example, the information processing device 100 iteratively performs a series of processes until a predetermined exit condition is satisfied. The series of processes includes, for example, a process of calculating a density matrix corresponding to each fragment based on a Lagrange multiplier using the first optimization calculation method. The series of processes includes, for example, a process of updating the Lagrangian multiplier so as to maintain consistency among the multiple fragments by using the second optimization calculation method when a predetermined exit condition is not satisfied.

Specifically, the information processing device 100 may communicate with a chemical calculating device 201 that performs the first optimization calculation method. Specifically, the information processing device 100 controls the chemical calculating device 201 to calculate the density matrix corresponding to each fragment based on the Lagrange multiplier. Specifically, the information processing device 100 may communicate with the chemical calculating device 201, which performs the second optimization calculation method. Specifically, the information processing device 100 controls the chemical calculating device 201 to refer to the Jacobian approximation formula and based on the calculated density matrices update the Lagrange multiplier according to the Newton-Krylov method so as to maintain the consistency between the fragments.

For example, when a predetermined exit condition is satisfied, the information processing device 100 calculates the energy of the molecule of interest based on the Lagrange multiplier last updated and the density matrix last calculated. Specifically, the information processing device 100 controls the chemical calculating device 201 to calculate the energy of the molecule of interest based on the Lagrange multiplier last updated and the density matrix last calculated. Specifically, the information processing device 100 receives the energy of the molecule of interest from the chemical calculating device 201.

The information processing device 100 outputs the calculated energy of the molecule of interest as a result of performing the quantum chemical calculation for the molecule of interest. The output format is, for example, display on a display, print output to a printer, transmission to another computer, or storage in a storage area. The other computer is, for example, the client device 202. The information processing device 100 may output the calculated energy of the molecule of interest as a result of performing the quantum chemical calculation for the molecule of interest, so that the user may refer to the energy. The information processing device 100 is, for example, a server or a PC.

The chemical calculating device 201 is a computer that performs quantum chemical calculations for molecules. The chemical calculating device 201 calculates a density matrix corresponding to each fragment based on the Lagrange multiplier using the first optimization calculation method under the control of the information processing device 100. For example, the chemical calculation device 201 may calculate the density matrix corresponding to each fragment in cooperation with another chemical calculation device 201.

Under the control of the information processing device 100, the chemical calculating device 201 uses the second optimization calculation method, refers to the Jacobian approximation formula and based on the density matrix, updates the Lagrange multiplier so as to maintain the consistency between the fragments. The update follows, for example, the Newton-Krylov method. For example, the chemical calculation device 201 may update the Lagrange multiplier in cooperation with another chemical calculation device 201.

The chemical calculating device 201, under the control of the information processing device 100, calculates the energy of the molecule of interest based on the Lagrange multiplier last updated and the density matrix last calculated. For example, the chemical calculating device 201 may calculate the energy of the molecule of interest in cooperation with another chemical calculating device 201. The chemical calculating device 201 transmits the energy of the molecule of interest to the information processing device 100. The chemical calculation device 201 is, for example, a server or a PC. The chemical calculation device 201 may be, for example, a quantum computer.

The client device 202 is a computer utilized by a user who desires to perform a quantum chemical calculation for a molecule of interest. The user is, for example, an operator. The client device 202 generates a processing request requesting execution of the quantum chemical calculation for the molecule of interest using the molecule dividing method, in response to the operation input of the user. The client device 202 obtains structure information indicating the structure of the molecule of interest, for example, in response to an operation input by the user. The client device 202 generates, for example, a processing request including structural information indicating the structure of the molecule of interest.

The client device 202 transmits the generated processing request to the information processing device 100. The client device 202 receives from the information processing device 100, a result of performing the quantum chemical calculation for the molecule of interest. The client device 202 outputs the result of performing the quantum chemical calculation for the molecule of interest so that the user may refer to the result. The client device 202 is, for example, a PC, a tablet terminal, or a smartphone.

Here, while a case in which the information processing device 100 is a device different from the chemical calculating device 201 has been described, the present disclosure is not limited hereto. For example, the information processing device 100 may have a function as the chemical calculating device 201 and may also operate as the chemical calculating device 201. In this case, the information processing system 200 may omit the chemical calculation device 201.

Here, while a case in which the information processing device 100 is a device different from the client device 202 has been described, the present disclosure is not limited hereto. For example, the information processing device 100 may have a function as the client device 202 and may also operate as the client device 202. In this case, the information processing system 200 may omit the client device 202.

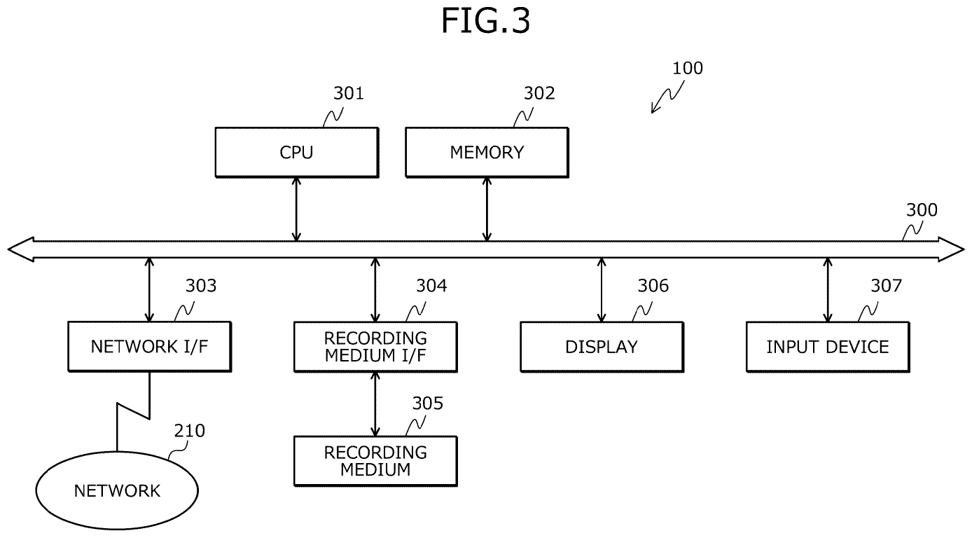

Next, with reference to FIG. 3, an example of a hardware configuration of the information processing device 100 is described.

FIG. 3 is a block diagram depicting an example of a hardware configuration of the information processing device 100. In FIG. 3, the information processing device 100 has a central processing unit (CPU) 301, a memory 302, and a network interface (I/F) 303. Further, the information processing device 100 has a recording medium I/F 304, a recording medium 305, a display 306, and an input device 307. Further, the components are coupled to each other by a bus 300.

Here, the CPU 301 governs overall control of the information processing device 100. The memory 302 includes, for example, a read-only memory (ROM), a random-access memory (RAM), a flash ROM, etc. In particular, for example, the flash ROM and the ROM store therein various programs and the RAM is used as a work area of the CPU 301. The programs stored in the memory 302 are loaded onto the CPU 301, whereby encoded processes are executed by the CPU 301.

The network I/F 303 is coupled to the network 210 through a communications line and communicates with other computers via the network 210. Further, the network I/F 303 administers an internal interface with the network 210 and controls the input and output of data from the other computers. The network I/F 303, for example, is a modem, a LAN adapter, etc.

The recording medium I/F 304, under the control of the CPU 301, controls the reading and writing of data with respect to the recording medium 305. The recording medium I/F 304 is, for example, a disk drive, a solid-state drive (SSD), a universal serial bus (USB) port, etc. The recording medium 305 is a nonvolatile memory storing therein data written thereto under the control of the recording medium I/F 304. The recording medium 305, for example, is a disk, a semiconductor memory, a USB memory, etc. The recording medium 305 may be removable from the information processing device 100.

The display 306 displays a cursor, icons, toolboxes, documents, images, or functional information, etc. The display 306, for example, is a cathode ray tube (CRT), a liquid crystal display, or an organic electroluminescence (EL) display, etc. The input device 307 has keys for inputting characters, numerals, or various instructions and performs data input. The input device 307, for example, is a keyboard or a mouse, etc. The input device 307, for example, may be a touch-panel input pad or numeric keypad.

In addition to the components above, the information processing device 100 may have, for example, a camera, etc. In addition to the components above, the information processing device 100 may have, for example, a printer, a scanner, a microphone, or a speaker, etc. Further, the information processing device 100, for example, may have the recording medium I/F 304 and the recording medium 305 in plural. Further, in the information processing device 100, for example, the display 306 and/or the input device 307, etc. may be omitted. Further, in the information processing device 100, for example, the recording medium I/F 304 and the recording medium 305 may be omitted.

An example of a hardware configuration example of the chemical calculating device 201 is, for example, similar to the example of the hardware configuration of the information processing device 100 depicted in FIG. 3 and thus, description thereof is omitted.

An example of a hardware configuration example of the client device 202 is, for example, similar to the example of the hardware configuration of the information processing device 100 depicted in FIG. 3 and thus, description thereof is omitted.

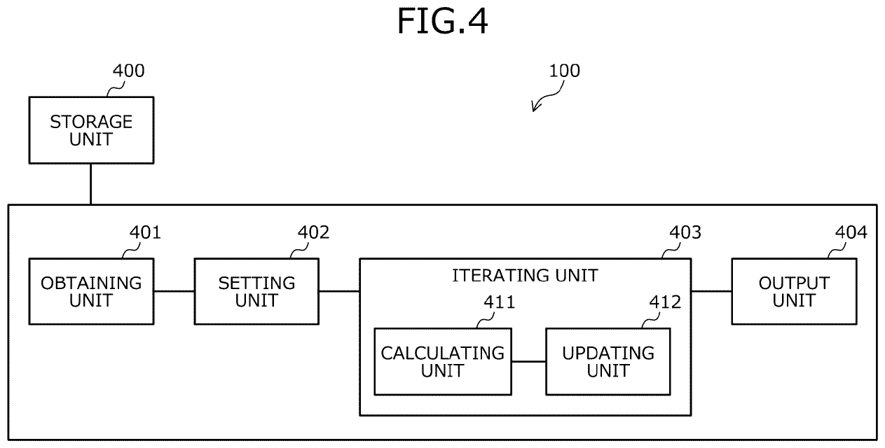

Next, an example of a functional configuration of the information processing device 100 will be described with reference to FIG. 4.

FIG. 4 is a block diagram depicting an example of the functional configuration of the information processing device 100. The information processing device 100 includes a storage unit 400, an obtaining unit 401, a setting unit 402, an iterating unit 403, and an output unit 404. The iterating unit 403 includes a calculating unit 411 and an updating unit 412.

The storage unit 400 is realized by, for example, a storage area such as the memory 302 or the recording medium 305 depicted in FIG. 3. Hereinafter, a case where the storage unit 400 is included in the information processing device 100 will be described, the present disclosure is not limited hereto. For example, the storage unit 400 may be included in a device different from the information processing device 100, and the storage content of the storage unit 400 may be referred to by the information processing device 100.

The obtaining unit 401 to the output unit 404 function as an example of a controller. Specifically, the functions of the obtaining unit 401 to the output unit 404 are realized, for example, by causing the CPU 301 to execute a program stored in a storage area such as the memory 302 or the recording medium 305 depicted in FIG. 3 or by the network I/F 303. The processing result of each functional unit is stored in, for example, a storage area such as the memory 302 or the recording medium 305 depicted in FIG. 3.

The storage unit 400 stores various types of information referred to or updated in the processes of the functional units. The storage unit 400 stores, for example, structural information indicating the structure of the molecule of interest. The molecule of interest includes multiple atoms. The structural information includes, for example, coordinates of each atom forming the molecule of interest. The structural information includes, for example, the type of each atom forming the molecule of interest. The structure information is obtained by, for example, the obtaining unit 401.

The storage unit 400 stores, for example, fragment information indicating each of multiple fragments obtained by dividing the structure of the molecule of interest. The fragment information includes, for example, coordinates of each atom of one or more atoms belonging to the fragment. The fragment information includes, for example, the type of each of the one or more atoms belonging to the fragment. Fragments share, for example, at least one atom. The fragment information is set by the setting unit 402, for example. The fragment information may be obtained by the obtaining unit 401, for example.

The storage unit 400 stores, for example, the first optimization calculation method. The first optimization calculation method is, for example, CCSD, FCI, VQE, or the like. The first optimization calculation method is set in advance by a user, for example. The first optimization calculation method may be obtained by the obtaining unit 401, for example.

The storage unit 400 stores, for example, the second optimization calculation method having a smaller calculation amount than the first optimization calculation method. The second optimization calculation method is, for example, a second-order Moller-Plesset method. The second optimization calculation method is set in advance by the user, for example. The second optimization calculation method may be obtained by the obtaining unit 401, for example.

The obtaining unit 401 obtains various types of information used for the processes of the functional units. The obtaining unit 401 stores the obtained various types of information to the storage unit 400 or outputs the obtained various types of information to the functional units. In addition, the obtaining unit 401 may output various types of information stored in the storage unit 400 to the functional units. The obtaining unit 401 obtains various types of information based on, for example, an operation input of a user. For example, the obtaining unit 401 may receive various types of information from a device different from the information processing device 100.

The obtaining unit 401 obtains, for example, a processing request requesting execution of quantum chemical calculation for the molecule of interest. The processing request may include, for example, structure information. The processing request may include, for example, fragment information. Specifically, the obtaining unit 401 obtains the processing request by receiving an input of the processing request. Specifically, the obtaining unit 401 may obtain the processing request by receiving the processing request from another computer. The other computer is, for example, the client device 202.

The obtaining unit 401 obtains, for example, structure information. Specifically, the obtaining unit 401 obtains the structure information by receiving an input of the structure information. Specifically, the obtaining unit 401 may obtain the structure information by receiving the structure information from another computer. The other computer is, for example, the client device 202. Specifically, the obtaining unit 401 may obtain the structure information by extracting the structure information from the processing request.

The obtaining unit 401 obtains fragment information, for example. Specifically, the obtaining unit 401 obtains the fragment information by receiving an input of the fragment information. Specifically, the obtaining unit 401 may obtain the fragment information by receiving the fragment information from another computer. The other computer is, for example, the client device 202. Specifically, the obtaining unit 401 may obtain the fragment information by extracting the fragment information from the processing request.

The obtaining unit 401 obtains, for example, the first optimization calculation method. Specifically, the obtaining unit 401 obtains the first optimization calculation method by receiving an input of the first optimization calculation method. Specifically, the obtaining unit 401 may obtain the first optimization calculation method by receiving the first optimization calculation method from another computer. The other computer is, for example, the client device 202.

The obtaining unit 401 obtains, for example, the second optimization calculation method. Specifically, the obtaining unit 401 obtains the second optimization calculation method by receiving an input of the second optimization calculation method. Specifically, the obtaining unit 401 may obtain the second optimization calculation method by receiving the second optimization calculation method from another computer. The other computer is, for example, the client device 202.

The obtaining unit 401 may receive a start trigger for starting the process of any functional unit. The start trigger is, for example, a predetermined operation input by the user. The start trigger may be, for example, reception of predetermined information from another computer. The start trigger may be, for example, output of predetermined information by any functional unit. Specifically, the obtaining unit 401 regards obtaining the processing request as a start trigger for starting the process of the iterating unit 403.

The setting unit 402 sets an initial value of the Lagrange multiplier. The initial value may be set in advance by the user, for example. Accordingly, the setting unit 402 may calculate a density matrix corresponding to each fragment.

The setting unit 402 sets fragment information indicating each of the multiple fragments obtained by dividing the structure of the molecule of interest. Fragments share, for example, at least one atom. For example, the setting unit 402 divides the structure of the molecule of interest into multiple fragments and sets fragment information indicating each fragment.

Specifically, the setting unit 402 divides the structure of the molecule of interest into multiple fragments so that each fragment includes an atom other than hydrogen and a hydrogen atom directly connected to the atom other than hydrogen. Accordingly, the setting unit 402 may reduce the workload on the user when setting the fragment information.

The iterating unit 403 iteratively performs a series of processes by the calculating unit 411 and the updating unit 412 until a predetermined exit condition is satisfied. The predetermined exit condition is set in advance by the user, for example. The predetermined exit condition is, for example, that the density matrices respectively corresponding to the fragments match or are similar. Accordingly, the iterating unit 403 may optimize the density matrix and the Lagrange multiplier.

The calculating unit 411 calculates a density matrix corresponding to each fragment based on the Lagrange multiplier by using the first optimization calculation method. For example, the calculating unit 411 obtains a wave function corresponding to each fragment by solving an unconstrained eigenvalue problem based on the Lagrange multiplier and calculates a density matrix corresponding to the fragment. Accordingly, the calculating unit 411 may optimize the density matrix corresponding to each fragment.

When the predetermined exit condition is not satisfied, the updating unit 412 updates the Lagrange multiplier so as to maintain consistency between the fragments based on the density matrices calculated by the calculating unit 411 and using the second optimization calculation method. Maintaining consistency means that the density matrices match or are similar for overlapping portions between the fragments.

The updating unit 412 uses, for example, the second optimization calculation method, refers to the Jacobian approximation formula and based on the density matrix, updates the Lagrange multiplier according to the Newton-Krylov method, so as to maintain the consistency between the fragments. Accordingly, the updating unit 412 may optimize the Lagrange multiplier. The updating unit 412 may reduce the amount of processing necessary to update the Lagrange multiplier, and may reduce the amount of processing necessary to perform the entire quantum chemical calculation.

When a predetermined exit condition is satisfied, the iterating unit 403 calculates the energy of the molecule based on the Lagrange multiplier last updated and the density matrix last calculated. Accordingly, the iterating unit 403 may accurately calculate the energy of the molecule based on the optimized Lagrange multiplier and the optimized density matrix, and may complete the quantum chemical calculation.

The output unit 404 outputs a process result of at least one of the functional units. The output format is, for example, display on a display, print output to a printer, transmission to an external device by the network I/F 303, or storage in a storage area such as the memory 302 or the recording medium 305. Accordingly, the output unit 404 may notify the user of the processing result of at least one of the functional units, and the convenience of the information processing device 100 may be improved.

For example, when the predetermined exit condition is satisfied, the output unit 404 outputs the Lagrange multiplier last updated and the density matrix last calculated. Specifically, the output unit 404 outputs the Lagrange multiplier last updated and the density matrix last calculated so that the user may refer thereto. Specifically, the output unit 404 may transmit the Lagrange multiplier last updated and the density matrix last calculated to another computer. The other computer is, for example, the chemical calculation device 201 or the client device 202. Thus, the output unit 404 may make the optimized Lagrange multiplier and density matrix available externally. The output 404 may allow the energy of the molecule of interest to be calculated externally.

For example, when a predetermined exit condition is satisfied, the output unit 404 outputs the energy of the molecule of interest calculated by the iterating unit 403 as a result of performing the quantum chemical calculation for the molecule of interest. Specifically, the output unit 404 outputs the energy of the molecule of interest so that the user may refer to the energy. Specifically, the output unit 404 may transmit the energy of the molecule of interest to another computer. The other computer is, for example, the client device 202. Thus, the output unit 404 may make the energy of the molecule of interest available externally.

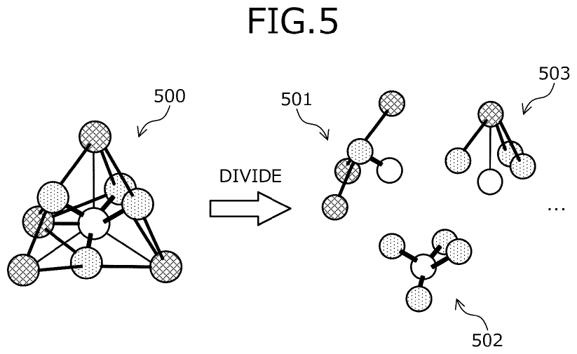

Next, an example of operation of the information processing device 100 will be described with reference to FIG. 5.

FIG. 5 is an explanatory diagram depicting an example of operation of the information processing device 100. In FIG. 5, the information processing device 100 divides the structure of a molecule of interest into multiple fragments and performs quantum chemical calculation for each fragment, thereby performing quantum chemical calculation for the entire molecule of interest and calculating the energy of the molecule of interest.

In the example depicted in FIG. 5, it is assumed that the information processing device 100 divides a structure 500 of a molecule of interest into fragments 501 to 503 and the like. When performing quantum chemical calculation for the molecule of interest, the information processing device 100 iteratively performs a first optimization calculation process of calculating a particle density matrix and a second optimization calculation process of maintaining consistency between fragments until a predetermined exit condition is satisfied.

In the following description, the particle density matrix may be referred to as “PDM”. In the following description, the first optimization calculation process may be referred to as “PDM calculation”. In the following description, the second optimization calculation process may be referred to as “PDM matching”.

In the PDM calculation, for example, a wave function YA corresponding to each fragment A is obtained. The wave function Y′A is defined by the following formula (1), for example. The constraint on the following formula (1) is defined by, for example, the following formulae (2) to (4).

$$\\ \begin{matrix}{Ψ}_{A} = arg {min}_{{Ψ}_{A}} {〈 \hat{H} {}_{emb}^{A} 〉}_{A} & ( 1 )\end{matrix}$$

$$\\ \begin{matrix}〈 {Ψ}_{A} \vert {Ψ}_{A} 〉 = 1 & ( 2 )\end{matrix}$$

$$\\ \begin{matrix}{〈 {a}_{p}^{†} {a}_{q} 〉}_{A} = {P}_{pq}^{B} & ( 3 )\end{matrix}$$

$$\\ \begin{matrix}∀ p , q ∈ {ℂ}_{B} ⋂ {𝔼}_{A} & ( 4 )\end{matrix}$$

Here, B is another fragment that overlaps with fragment A. Fragment B shares at least one atom with fragment A, for example. H_{emb}^{A} with circumflex “A” thereabove is the Hamiltonian of fragment A. PB is 1PDM of fragment B. aₚ† and a_{q} are a generation operator and an annihilation operator, respectively. C_{B} and Eₐ are the central site of the fragment B and the edge site of the fragment A, respectively. < . . . >_{AS}:=<ψ_{A}| . . . |ψ_{A}>.

Formulae (1) to (4) are constrained optimization problems. Therefore, the above formulae (1) to (4) may be reduced to an unconstrained eigenvalue problem based on the Lagrange multiplier λ. The Lagrange multiplier λ is defined by the following formula (5). The unconstrained eigenvalue problem is defined by the following formulae (6) and (7).

$$\\ \begin{matrix}λ = {( {λ}_{p , q}^{A} )}_{p , q ∈ {C}_{A} ⋂ {E}_{B ,} A \neq B} & ( 5 )\end{matrix}$$

$$\\ \begin{matrix}( \hat{H} {}_{emb}^{A} + {\hat{λ}}_{A} ) \vert {Ψ}_{A} 〉 = {ℰ}_{A} \vert {Ψ}_{A} 〉 & ( 6 )\end{matrix}$$

$$\\ \begin{matrix}{\hat{λ}}_{A} = {\sum }_{B \neq A} [ {\sum }_{p , q ∈ {ℂ}_{B} ⋂ {𝔼}_{A}} {λ}_{pq}^{B} {a}_{p}^{†} {a}_{q} ] + μ {\sum }_{p ∈ {ℂ}_{A}} {a}_{p}^{†} {a}_{p} & ( 7 )\end{matrix}$$

By solving the eigenvalue problem for the set Lagrange multipliers, the wave function ψ_{A} corresponding to each fragment A may be obtained, and PDMP_{A} may be calculated. For example, PDMP_{A} may be calculated based on the solution of the above formula (6) in the case of λ_{A}=0 with circumflex “\hat{ }” thereover. Therefore, the PDM calculation is realized by solving the eigenvalue problem for the set Lagrange multiplier. The eigenvalue problem may be solved by, for example, an optimization calculation method. Specifically, the eigenvalue problem may be solved by the first optimization calculation method with relatively high accuracy such as CCSD, FCI, or VQE.

In the PDM matching, the Lagrange multiplier (λ_{pq}^{A}) is optimized so that ε_{BE} becomes 0. Here, ε_{BE} is defined by the following formula (8). The vector value function F is defined by the following formula (9). The Lagrange multiplier may be updated by solving the vector-valued function F(λ)=0. The vector-valued function F(λ) is R^{d}→R^{d}. The vector value function F depends on the optimization calculation method.

$$\\ \begin{matrix}{ε}_{BE} = {[ \frac{1}{{N}_{cons}} {\sum }_{A} {\sum }_{B \neq A} {\sum }_{p , q ∈ {ℂ}_{A} ⋂ {𝔼}_{B}} {( {P}_{pq}^{A} - {P}_{pq}^{B} )}^{2} ]}^{1 / 2} & ( 8 )\end{matrix}$$

$$\\ \begin{matrix}F : λ = {( {λ}_{p , q}^{A} )}_{p , q ∈ {C}_{A} ⋂ {E}_{B ,} A \neq B} ↦ {( {P}_{p , q}^{A} ( λ ) - {P}_{p , q}^{B} ( λ ) )}_{p , q ∈ {C}_{A} ⋂ {E}_{B ,} A \neq B} & ( 9 )\end{matrix}$$

Conventionally, there is a method of performing PDM matching by optimizing a Lagrange multiplier (λ_{pq}^{A}) so that ε_{BE} becomes 0 by the Newton method. In this method, specifically, linear equations defined by the following formulae (10) and (11) are solved for each iteration of the Newton method. Here, k=0, 1, . . . u corresponds to λ. J represents the Jacobian of the vector-valued function F. As the number of dimensions of u increases, when the Jacobian is calculated by the finite difference method, the difference is calculated in all directions of u, and there is a problem that the processing amount necessary when the PDM matching is performed increases.

$$\\ \begin{matrix}J ( {u}^{k} ) δ {u}^{k} = - F ( {u}^{k} ) & ( 10 )\end{matrix}$$

$$\\ \begin{matrix}{u}^{k + 1} = {u}^{k} + δ {u}^{k} & ( 11 )\end{matrix}$$

In addition, there is a method of performing PDM matching by optimizing a Lagrange multiplier (λ_{pq}^{A}) so that ε_{BE} becomes 0 by the Newton-Krylov method. In this method, specifically, a Jacobian approximation formula defined by the following formula (12) is calculated. Therefore, Jv may be calculated for a specific directional vector v for each iteration of the Newton-Krylov method. Here, when the first optimization calculation method with relatively high accuracy such as CCSD, FCI, or VQE is performed in order to calculate the vector value function F, there is a problem in that an increase in the processing amount is caused when PDM matching is performed.

$$\\ \begin{matrix}Jv \approx [ F ( u + ϵ v ) - F ( u ) ] / ϵ & ( 12 )\end{matrix}$$

Therefore, the information processing device 100 calculates a function F with “~” thereabove, which is a substitute for the vector-valued function F, using the second optimization calculation method such as the Moller-Plesset method, which tends to have a smaller amount of calculation than CCSD, FCI, VQE, or the like. The information processing device 100 performs PDM matching by calculating a Jacobian approximation formula defined by the following formula (13) based on a function F with “~” thereabove. As a result, the information processing device 100 may reduce the amount of processing necessary to perform PDM matching and reduce the amount of processing necessary to perform the entire quantum chemical calculation as compared with related arts.

$$\\ \begin{matrix}Jv \approx ( \tilde{F} ( u + ϵ v ) - \tilde{F} ( u ) ) / ϵ & ( 13 )\end{matrix}$$

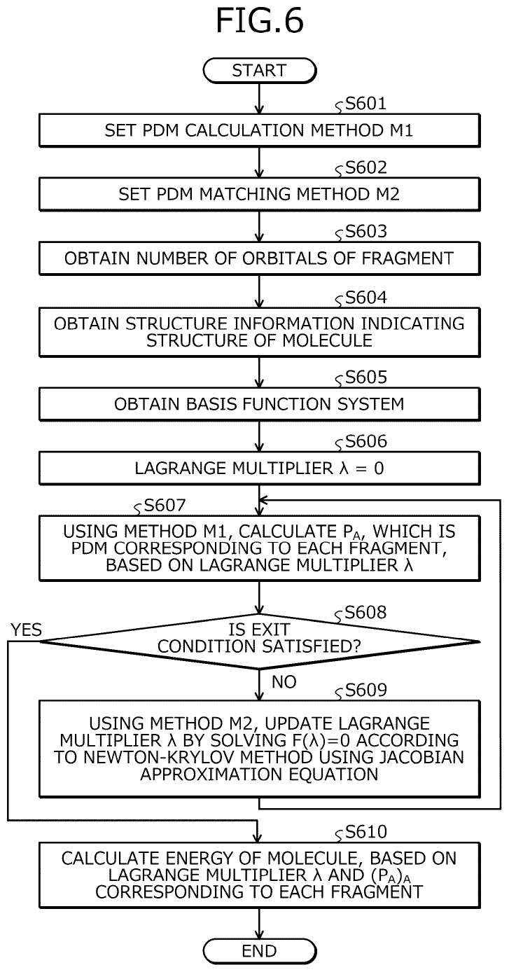

Next, an example of a procedure of an overall process executed by the information processing device 100 will be described with reference to FIG. 6. The overall process is implemented by, for example, the CPU 301, storage areas such as the memory 302 and the recording medium 305, and the network I/F 303 depicted in FIG. 3.

FIG. 6 is a flowchart depicting an example of the procedure of the overall process. In FIG. 6, the information processing device 100 sets a PDM calculation method M1 (step S601). The information processing device 100 sets a PDM matching method M2 (step S602).

The information processing device 100 obtains the number of orbitals of the fragment (step S603). The information processing device 100 obtains structure information indicating the structure of a molecule (step S604). The information processing device 100 obtains a basis function system (step S605).

The information processing device 100 performs an initialization process to set the Lagrange multiplier λ to 0 (step S606). Using the method M1, the information processing device 100 calculates P_{A}, which is a PDM corresponding to each fragment, based on the Lagrange multiplier λ (step S607).

The information processing device 100 determines whether an exit condition is satisfied (step S608). Here, when the exit condition is not satisfied (step S608: NO), the information processing device 100 proceeds to the process at step S609. On the other hand, when the exit condition is satisfied (step S608: YES), the information processing device 100 proceeds to the process at step S610.

At step S609, the information processing device 100, using the method M2, updates the Lagrange multiplier λ by solving F(λ)=0 according to the Newton-Krylov method using the Jacobian approximation formula (step S609). The information processing device 100 returns to the process at step S607.

At step S610, the information processing device 100 calculates the energy of the molecule, based on the Lagrange multiplier λ and (P_{A})_{A} corresponding to each fragment (step S610). The information processing device 100 ends the entire process. Accordingly, the information processing device 100 may complete the quantum chemical calculation while reducing the processing amount necessary to perform the quantum chemical calculation.

Here, the information processing device 100 may change the sequence of the processes of some steps in FIG. 6 and execute the processes. For example, the sequence of the processes of steps S601 to S605 may be interchanged. In addition, the information processing device 100 may omit the processes of some steps in FIG. 6. For example, the process at step S610 may be omitted.

The information processing device 100 may be applied to fields such as drug discovery and material development. Specifically, in the field of drug discovery, material development, or the like, the information processing device 100 may be applied to a case where it is desired to perform quantum chemical calculation for calculating the basis energy of a molecule in order to analyze the structure or properties of the molecule that is a candidate for a drug or a material. As a result, the information processing device 100 may maintain the accuracy of the quantum chemical calculation while reducing the amount of processing necessary to perform the quantum chemical calculation, may facilitate calculation of the basis energy of a molecule, and may contribute to the fields of drug discovery, material development, and the like.

As described above, according to the information processing device 100, it is possible to use the first optimization calculation method and the second optimization calculation method having a smaller calculation amount than the first optimization calculation method. According to the information processing device 100, it is possible to use the first optimization calculation method and calculate a density matrix corresponding to each of multiple fragments obtained by dividing the structure of the molecule, based on the predetermined parameter. According to the information processing device 100, in a case where the predetermined exit condition is not satisfied, it is possible to use the second optimization calculation method, refer to the Jacobian approximation formula, and update the predetermined parameter so as to maintain the consistency between the fragments, based on the density matrix. According to the information processing device 100, the process of calculating a density matrix corresponding to each fragment and the process of updating the predetermined parameter may be iteratively executed until the predetermined exit condition is satisfied. Accordingly, the information processing device 100 may reduce the amount of processing necessary to perform optimization calculation for updating a predetermined parameter and reduce the amount of processing necessary to perform the entire quantum chemical calculation. Further, the information processing device 100 may optimize a density matrix corresponding to each fragment and a predetermined parameter.

According to the information processing device 100, when the predetermined exit condition is satisfied, the energy of the molecule may be calculated based on the predetermined parameter last updated and the density matrix last calculated. Thus, the information processing device 100 may complete the quantum chemical calculation.

According to the information processing device 100, it is possible to set CCSD, FCI, or VQE in the first optimization calculation method. According to the information processing device 100, it is possible to set the second-order Moller-Plesset method as the second optimization calculation method. Accordingly, the information processing device 100 may reduce the amount of processing necessary to perform optimization calculation for updating the predetermined parameter and reduce the amount of processing necessary to perform quantum chemical calculation while maintaining the accuracy of calculating the energy of a molecule.

According to the information processing device 100, it is possible to calculate the density matrix corresponding to each fragment of the multiple fragments obtained by dividing the structure of the molecule such that the fragments share at least one or more atoms forming the molecule. According to the information processing device 100, when the predetermined exit condition is not satisfied, it is possible use the second optimization calculation method to update the predetermined parameter in a direction so that the calculated density matrices coincide with each other with respect to at least one or more atoms shared by fragments. Accordingly, the information processing device 100 may easily update the predetermined parameter with high accuracy.

According to the information processing device 100, the calculated energy of the molecule may be output. Accordingly, the information processing device 100 may enable the result of performing the quantum chemical calculation to be referred to externally.

The information processing method described in the present embodiment may be implemented by executing a prepared program on a computer such as a personal computer and a workstation. The program is stored on a non-transitory, computer-readable recording medium such as a hard disk, a flexible disk, a compact disc read-only memory (CD-ROM), a magneto-optical (MO) disc, and a digital versatile disc (DVD), read out from the computer-readable medium, and executed by the computer. The program may be distributed through a network such as the Internet.

According to one aspect, it is possible to easily reduce the amount of processing necessary to perform optimization calculations.

All examples and conditional language provided herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A computer-readable recording medium storing therein a program for causing a computer to execute a process, the process comprising:
calculating a plurality of density matrices respectively corresponding to a plurality of fragments obtained by dividing a structure of a molecule, based on a predetermined parameter by using a first optimization calculation method; and
updating the predetermined parameter according to a Newton-Krylov method so as to maintain consistency among the plurality of fragments, when a predetermined exit condition is not satisfied, the predetermined parameter being updated based on the calculated plurality of density matrices and by referring to a Jacobian approximation formula and using a second optimization calculation method having a smaller calculation amount than the first optimization calculation method, wherein
the calculating and updating are iteratively executed until the predetermined exit condition is satisfied.

2. The computer-readable recording medium according to claim 1, the process further comprising when the predetermined exit condition is satisfied, calculating an energy of the molecule based on the predetermined parameter last updated and the plurality of density matrices last calculated.

3. The computer-readable recording medium according to claim 1, wherein
the first optimization calculation method is any one of a coupled-cluster calculation with single and double excitations, a full configuration interaction, and a variational quantum Eigen-solver, and
the second optimization calculation method is a second-order Moller-Plesset method.

4. The computer-readable recording medium according to claim 3, wherein
fragments of the plurality of fragments share at least one or more atoms forming the molecule, and
the updating, when the predetermined exit condition is not satisfied, includes using the second optimization calculation method, referring to the Jacobian approximation formula, and according to the Newton-Krylov method, updating the predetermined parameter in a direction so that the calculated plurality of density matrices coincide with each other with respect to the at least one or more atoms shared by the fragments of the plurality of fragments.

5. The computer-readable recording medium according to claim 1, the process further comprising outputting the calculated energy of the molecule.

6. The computer-readable recording medium according to claim 1, wherein the predetermined parameter includes a Lagrange multiplier.

7. The computer-readable recording medium according to claim 1, wherein the predetermined parameter includes a chemical potential u.

8. An information processing method executed by a computer, the method comprising:
calculating a plurality of density matrices respectively corresponding to a plurality of fragments obtained by dividing a structure of a molecule, based on a predetermined parameter by using a first optimization calculation method; and
updating the predetermined parameter according to a Newton-Krylov method so as to maintain consistency among the plurality of fragments, when a predetermined exit condition is not satisfied, the predetermined parameter being updated based on the calculated plurality of density matrices and by referring to a Jacobian approximation formula and using a second optimization calculation method having a smaller calculation amount than the first optimization calculation method, wherein
the calculating and updating are iteratively executed until the predetermined exit condition is satisfied.

9. An information processing device comprising:
a memory; and
a processor coupled to the memory, the processor configured to:
calculate a plurality of density matrices respectively corresponding to a plurality of fragments obtained by dividing a structure of a molecule, based on a predetermined parameter by using a first optimization calculation method; and
update the predetermined parameter according to a Newton-Krylov method so as to maintain consistency among the plurality of fragments, when a predetermined exit condition is not satisfied, the predetermined parameter being updated based on the calculated plurality of density matrices and by referring to a Jacobian approximation formula and using a second optimization calculation method having a smaller calculation amount than the first optimization calculation method, wherein
calculation of the plurality of density matrices and updating of the predetermined parameter are iteratively executed until the predetermined exit condition is satisfied.
